SEQUENCE LISTING (6 sequences)

SEQ ID NO: 1 - DNA, 849 bp, Human
  misc_feature (792)..(792): n is a, c, g, or t
cggcccagccggccatggccgaagttcagctgcagcagtctggactgaactggtgaagcctggggcttcagtgaagttgtcatgcaaggcttctggctacactttcaccagcgactggatgcactgggtgaagcagaggcctggacaaggccttgagtggattggaatgattcatccttatagtggtgatattaagttcaattgagaggttcaggaacaaggccacactgactgtagacaaatcctccaacacagcctatatgcaagtcagcagcctgacatctgaggactctgcggtctattactgtgcaagagatgatgattccgcctggtttgcttactggggccaagggactctggtcactgtctcttcgggaggtggtggatccggcggtggcggttctggtggaggtggatctrgatgttgtgatgacccagactccactctccctgcctgtcagtcttggagatcaagcctccatctcttgcagatctagtcagagcattgtacatagtaatggaaacacctatttagaatggtaccttcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggagggaccaagctggaactgaaacgggcggccgcacatcatcatcaccatcacgggggnccgcanaacaaaactcatctcaaaaaaggatctgaatggggccncatanactgttga

SEQ ID NO: 2 - DNA, 57 bp, Human
gcggccgcaggtgcgccggtgccgtatccggatccgctggaaccgcgtgccgcatag

SEQ ID NO: 3 - protein, 277 aa, Human
MAEVQLQQSGTELVKPGASVKLSCKASGYTFTSDWMHWVKQRPGQGLEWIGMIHPYSGDIKFNERFRNKATLTVDKSSNTAYMQVSSLTSEDSAVYYCARDDDSAWFAYWGQGTLVTVSSGGGGSGGGGSGGGGSDVVMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLELKRAAAHISHHHHHHGGPNKTHLKKGSEWGIC

SEQ ID NO: 4 - DNA, 33 bp, HUman
cgcggatccgttggagataaacttcctgaatgt

SEQ ID NO: 5 - DNA, 36 bp, Human
gcggaattcttaaatctcggggggcttcgggcagcc

SEQ ID NO: 6 - DNA, 95 bp, Human
gaattcgattattcttaaatctcggggggcttcgggcagccgtcatctgcttcacattcaggaagtttatctccaacggatccgcgaatcactag

(19) United States
(12) Patent Application Publication
(10) Pub. No.: US 2006/0228753 A1
(43) Pub. Date: Oct. 12, 2006

(54) Methods of detecting a phenotype of a polymorphic protein

(76) Inventors: Andrew Levy, Kiryat Shmuel (IL); Nina Levy, Haifa (IL)

Correspondence Address: PEARL COHEN ZEDEK, LLP, 1500 BROADWAY 12TH FLOOR, NEW YORK, NY, 10036, US

(21) Appl. No.: 11/177,495

(22) Filed: Jul. 11, 2005

Related U.S. Application Data
(60) Provisional application No. 60/586,733, filed on Jul. 12, 2004.

(51) Int. Cl. C07K 16/18; G01N 33/53; C07H 21/04; C12P 21/06; C12N 5/06

(52) U.S. Cl. 435/7.1; 435/69.1; 435/320.1; 435/326; 530/388.25; 536/23.53

(57) ABSTRACT

The present invention provides an antibody that differentially reacts with allelic variants of a polymorphic protein, methods of identifying same, an antigen binding fragment comprised therein, nucleic acids encoding same, proteins, cells, viral particles, compositions, and kits comprising same. The invention also provides methods for determining a haptoglobin type of a subject and methods for testing a subject for susceptibility to diabetic complications.

### CROSS REFERENCE TO RELATED APPLICATIONS

This application claims priority of U.S. Provisional Application No. 60/586,733, filed Jul. 12, 2004, which is incorporated hereby by reference in its entirety.

### FIELD OF THE INVENTION

The present invention provides an antibody that differentially reacts with allelic variants of a polymorphic protein, methods of identifying same, an antigen binding fragment comprised therein, nucleic acids encoding same, proteins, cells, viral particles, compositions, and kits comprising same. The invention also provides methods for determining a haptoglobin type of a subject and methods for testing a subject for susceptibility to diabetic complications.

### BACKGROUND OF THE INVENTION

The haptoglobin genetic locus at 16q22 is polymorphic with two known classes of alleles denoted 1 and 2 [Langlois M et al, Clin Chem 42: 1589-1600, 1996]. The polymorphism is quite common, with worldwide frequencies of the two alleles being approximately equal. Haptoglobin is a major susceptibility gene for the development of diabetic vascular complications in multiple longitudinal and cross-sectional population studies [Levy A et al, New Eng J Med 343: 969-70, 2000; Roguin A et al, Am J Card 87: 330-2, 2001]. Diabetic individuals homozygous for the haptoglobin 2 (Hp 2) allele are at 5 times greater risk of developing cardiovascular disease as compared to diabetic individuals homozygous for the haptoglobin 1 allele (Hp 1), with an intermediate risk present in the heterozygote Levy A et al, J Am Coll Card 40: 1984-90, 2002]. Mechanistic studies using the purified protein products of the Hp 1 and Hp 2 alleles have identified profound differences in antioxidant and immunomodulatory activity [Frank M et al, Blood; 98: 3693-8, 2001; Asleh R et al, Circ Res 92: 1193-200, 2003].

Functional as well as structural differences exist between the various haptoglobin allelic protein products [[anglois M et al, Clin Chem 42: 1589-1600, 1996]. The Hp 2 allele has two copies of exon 3 and 4 of the Hp1 allele, which results in the duplication of a multimerization domain in exon 3. Consequently, while the Hp1 allele protein product forms only dimers, Hp2 allele protein products combine to form cyclic polymers ranging in size from three monomers and upwards. In heterozygotes, linear polymers containing both allelic protein products are present.

The development of an antibody based ELISA test to type haptoglobin has been hampered by the apparent lack of antigenic determinants unique to either allelic protein product. Apart from a single junction at the site of duplication of exon three, there exist no differences in primary amino acid sequence between the haptoglobin alleles. Given the need to screen large populations of diabetic individuals (10% of the western world) for their haptoglobin type in order to determine optimal treatment as well as the need to screen certain populations rapidly (i.e. individuals suffering from an acute myocardial infarction) there is a great need for a simple, rapid, inexpensive test for haptoglobin typing.

### SUMMARY OF THE INVENTION

In one embodiment, the present invention provides an anti-haptoglobin (Hp) antibody that binds with greater affinity to Hp 2-2 than to Hp 2-1, and with greater affinity to Hp 2-1 than to Hp 1-1. In one embodiment, the antibody may have an amino acid sequence as set forth in SEQ ID No 1.

In another embodiment, the present invention provides an antigen binding fragment of an anti-haptoglobin (Hp) antibody that binds with greater affinity to a first haptoglobin isoform than to a second haptoglobin isoform. In another embodiment, the present invention provides an antibody or recombinant protein comprising the antigen binding fragment. In one embodiment, the antibody may be monoclonal. In another embodiment, the antibody may be polyclonal. In another embodiment, the antibody may be humanized or chimeric. In another embodiment, the antibody may be an scFv antibody.

In another embodiment, the present invention provides an isolated nucleic acid encoding any anti-haptoglobin (Hp) antibody of the present invention. In another embodiment, the present invention provides an isolated nucleic acid encoding any antigen-binding fragment of the present invention.

In another embodiment, the present invention provides a method of determining a haptoglobin type of a subject, comprising (a) contacting a biological sample of the subject with an anti-haptoglobin antibody; and (b) quantitatively determining a binding or interaction between the haptoglobin protein and the antibody under conditions whereby a value obtained from the quantitatively determination is characteristic of a presence of Hp 1-1, Hp 2-1, or Hp 2-2 in the biological sample.

In another embodiment, any method of the present invention may be utilized to test a subject for susceptibility to diabetic complications.

In another embodiment, the present invention provides a method of testing an antibody or recombinant protein for a utility in distinguishing between Hp 1-1, Hp 2-1, and Hp 2-2, comprising (a) contacting a first quantity of the antibody or recombinant protein with an Hp 1-1 molecule; (b) contacting a second quantity of the antibody or recombinant protein with an Hp 2-1 molecule; (c) contacting a third quantity of the antibody or recombinant protein with an Hp 2-2 molecule; and (d) quantitatively determining a binding or interaction between the antibody or recombinant protein and the Hp 1-1, Hp 2-1, and Hp 2-2, whereby a value obtained from the quantitatively determination that is characteristic of the presence of each of Hp Hp 1-1, Hp 2-1, or Hp 2-2 indicates that the antibody distinguishes between Hp 1-1, Hp 2-1, and Hp 2-2.

In another embodiment, the present invention provides a method of testing an antibody or recombinant protein for a utility in distinguishing between Hp 1-1, Hp 2-1, and Hp 2-2, comprising (a) immobilizing an anti-haptoglobin antibody on a substrate to form an antibody-substrate complex; (b) contacting a first quantity of the antibody-substrate complex with an Hp 1-1 molecule; (c) contacting a second quantity of the antibody-substrate complex with an Hp 2-1 molecule; (d) contacting a third quantity of the antibody-substrate complex with an Hp 2-2 molecule; (e) contacting the products of steps (b), (c), and (d) with the test antibody or recombinant protein; and (e) quantitatively determining; a binding or interaction between the test antibody or recombinant protein and the Hp 1-1, Hp 2-1 and Hp 2-2; whereby a value obtained from the quantitatively determining that is characteristic of the presence of each of Hp 1-1, Hp 2-1 or Hp 2-2 indicates that the test antibody distinguishes between Hp 1-1, Hp 2-1 and Hp 2-2.

In another embodiment, the present invention provides a method of screening a plurality of test antibodies for an ability to differentially interact with different haptoglobin types, comprising (a) generating a plurality of vehicles, each comprising an antibody from the plurality of test antibodies and a nucleic acid molecule encoding the antibody; (b) contacting the plurality of vehicles with non-immobilized Hp 1-1 or Hp 2-1 and Hp 2-2 that is immobilized on a substrate; (c) subcloning a nucleic acid molecule from one of the plurality of vehicles into a vehicle that expresses an antibody encoded by the nucleic acid molecule; (d) repeating steps (b) and (c) one or more times; and (e) identifying an antibody or nucleic acid molecule present in a vehicle retained on the substrate.

In another embodiment, the present invention provides a method of distinguishing between two allelic variants of a polymorphic protein in a biological sample, wherein the two allelic variants differ in a number of copies of an epitope, comprising (a) contacting a biological sample with an antibody or recombinant protein, wherein the antibody or recombinant protein binds the polymorphic protein; and (b) quantitatively assessing a binding or interaction between the polymorphic protein and the antibody or recombinant protein; under conditions whereby the presence of each of the two allelic variants results in a value obtained from the quantitatively assessing that is characteristic of the allelic variant.

### BRIEF DESCRIPTION OF THE DRAWINGS

FIG. 1A-B. (A) Annotated sequence of e3 antibody with His and Myc tags. The Sfi I-Not I fragment was subcloned into pCANTAB6 to generate the His- and Myc-tagged antibody. Superscript notes the boundaries for the Sfi I and Not I sites, myc and tags, as -well as the sequence of the linker region linking the V_{H} and V_{K} chains that make up the single chain antibody. The sequence of the V_{H} chain is from the Sfi I site to the linker and the sequence of the V_{K} chain is from the linker to the Not I site. (B) The Sfi I-Not I fragment was subcloned into pCANTAB5e which replaces the his-myc tag with an Etag.

FIG. 2. E3 amino acid sequence of e3 antibody with His and Myc tags. The sequence begins with the V_{H} region, followed by the linker region linking the V_{H} and V_{K} chains, followed by the Not I site and His and Myc tags (each denoted by superscript). The amino acid sequence of the E-tagged construct is identical, except for the replacement of the His and Myc tags with the Etag.

FIG. 3. Ability of E3 antibody to distinguish between haptoglobin types is independent of haptoglobin concentration.

FIG. 4 shows a schematic diagram of the exon structure of the Hp gene(1 or 2 allele).

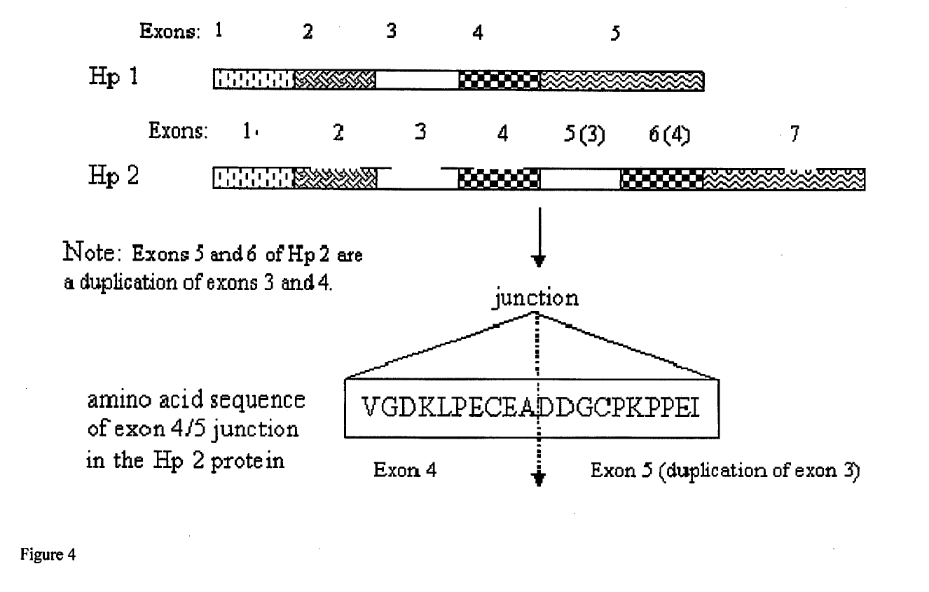

FIG. 5 show that the mouse fusion protein antiserum easily distinguishes between Hp 1-I and Hp 2-2.

FIG. 6 shows crude and affinity purified 4/5 junction peptide antiserum from rabbits is able to differentiate Hp 1-1, Hp 2-1 and Hp 2-2 in an ELISA format

### DETAILED DESCRIPTION OF THE PRESENT INVENTION

In one embodiment, the present invention provides an anti-haptoglobin (Hp) antibody that binds with greater affinity to Hp 2-2 than to Hp 2-1, and with greater affinity to Hp 2-1 than to Hp 1-1. Hp 2-2 refers, in one embodiment, to polymers of haptoglobin comprising Hp 2 but no Hp 1. Hp 2-1 refers, in one embodiment, to polymers of haptoglobin comprising both Hp 1 and Hp 2. Hp 1-1 refers, in one embodiment, to polymers of haptoglobin comprising Hp 1 but no Hp 2. In one embodiment, the antibody has an amino acid sequence as set forth in SEQ ID No 1.

In one embodiment, the antibody of the present invention is a monoclonal antibody. In another embodiment, the antibody of the present invention is a polyclonal antibody. The term “monoclonal antibody” (mAb) refers, in one embodiment, to an antibody obtained from a population of substantially homogeneous antibodies, i.e., the individual antibodies comprising the population are identical except for possible naturally occurring mutations that may be present in minor amounts. Monoclonal antibodies may be highly specific, directed against a single antigenic site. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al, Nature 256: 495 (1975), or may be made by recombinant DNA methods (see, e.g., U.S. Pat. No. 4,816,567). The “monoclonal antibodies” also include clones of antigen-recognition and binding-site containing antibody fragments (Fv clones) isolated from phage antibody libraries using the techniques described in Clackson et al., Nature, 352:624-628 (1991) and Marks et al., J. Mol. Biol., 222:581-597 (1991), for example. Each type of antibody represents a separate embodiment of the present invention.

The monoclonal antibodies herein include hybrid and recombinant antibodies 5 produced by splicing a variable (including hypervariable) domain of an antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′).sub.2, and Fv), so long as they exhibit the desired biological activity. (See, e.g., U.S. Pat. No. 4,816,567; Mage and Lamoyi, in Monoclonal Antibody Production Techniques and Applications, pp. 79-97, Marcel Dekker, Inc., New York, 1987). Variable and constant regions of antibodies are described below. Each type of antibody represents a separate embodiment of the present invention.

The monoclonal antibodies of the present invention include, in one embodiment, “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al., supra; Morrison et al., Proc. Natl. Acad. Sci. U.S.A. 81:6851, 1984). Each type of antibody represents a separate embodiment of the present invention.

“Humanized” forms of non-human (e.g., murine) antibodies are specific chimeric immunoglobulins, immunoglobulin chains or fragments thereof (such as Fv, Fab, Fab′, F(ab′).sub.2, or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Furthermore, humanized antibodies can comprise residues which are found neither in the recipient antibody nor in the imported CDR or framework sequences. These modifications are made s to further refine and maximize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature 321: 522, 1986; Reichmann et al., Nature 332: 323, 1988; Presta, Curr. Op. Struct. Biol. 2: 593, 1992).

Native antibodies are heterotetrameric glycoproteins of about 150,000 daltons, composed of two identical light (L) chains and two identical heavy (H) chains, comprising both intrachain and interchain disulfide bridges. Each heavy chain has at one end a variable domain (V.sub.H) followed by a number of constant domains. Each light chain has a variable domain at one end (V.sub.L) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a .beta.-sheet configuration, connected by three CDRs, 20 which form loops connecting, and in some cases forming part of, the .beta.-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md., 1991). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′).sub-2 fragment that has two antigen-combining sites and is still capable of cross-linking antigen. Each type of antibody fragment represents a separate embodiment of the present invention.

In one embodiment, the antibody of the invention is a single-chain Fv (scFv) antibody. “Fv” is, in one embodiment, the minimum antibody fragment which contains a complete antigen-recognition and -binding site, and is also referred to as a “antigen binding fragment” In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv, see Pluckthun, in The Pharmacology of Monoclonal Antibodies, vol. 113, Rosenburg and Moore eds., Springer-Verlag, New York, pp. 269-315 (1994).

The Fab fragment also contains the constant domain of the light chain and the first constant domain (CHI) of the heavy chain. Fab′ fragments differ from Fab fragments by the addition of a few residues at the carboxy terminus of the heavy chain CH1 domain including one or more cysteines from the antibody hinge region. Fab′ also represent an embodiment of the present invention.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG.sub.1, IgG.sub.2, IgG.sub.3, IgG.sub.4, IgA.sub.1, and IgA.sub.2. The heavy-chain constant domains that correspond to the different classes of immunoglobulins are called .alpha., .delta., .epsilon., .dwnarw., and .mu., respectively. The subunit structures and three-dimensional configurations of different classes of immunoglobulins are well known. The “light chains” of antibodies (immunoglobulins) from any vertebrate species can be assigned to one of two types, called kappa (k) and lambda (l), based on the amino acid sequences of their constant domains. Each type of antibody represents a separate embodiment of the present invention.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′).sub.2, and Fv fragments; diabodies (a class of small bivalent and bispecific antibody fragments; Proc Natl Acad Sci U S A 90: 6444-8, 1993); any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain s variable region, without an associated light chain moiety; and multispecific or multivalent structures formed from antibody fragments. Methods of determining the CDRs of an antibody are well known in the art, and are described, for example, in U.S. Pat. Nos. 6,750,325 and 6,632,926. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). Suitable leucine zipper sequences include the jun and fos leucine zippers taught by Kostelney et al., J. Immunol., 148: 1547-1553 (1992).

The term “antibody” as used herein, refers, in one embodiment, to any type of antibody or antibody fragment of the present invention, and to any type of antibody or antibody fragment known in the art.

In another embodiment, the present invention provides an antigen binding fragment of an anti-haptoglobin (Hp) antibody that binds with greater affinity to a first haptoglobin isoform than to a second haptoglobin isoform. In another embodiment, the present invention provides an antibody or recombinant protein comprising the antigen binding fragment of a anti-Hp antibody of the invention. In another embodiment, the present invention provides an antibody or recombinant protein comprising the CDR of a anti-Hp antibody of the invention. In one embodiment, the antibody may be monoclonal. In another embodiment, the antibody may be polyclonal. In another embodiment, the antibody may be humanized or chimeric. In another embodiment, the antibody may be an scFv antibody.

The present invention encompasses antibody variants of antibodies described herein. Antibody variant refers, in one embodiment, to an antibody that has an amino acid sequence that differs from the amino acid sequence of a parent antibody. Preferably, the antibody variant comprises a heavy chain variable domain or a light chain variable domain having an amino acid sequence that is not found in nature. Such variants necessarily have less than 100% sequence identity or similarity with the parent antibody. In one embodiment, the antibody variant will have an amino acid sequence having about 75% amino acid sequence identity or similarity with the amino acid sequence of either the heavy or light chain variable domain of the parent antibody. In another embodiment, the antibody variant will have about 77% sequence identity or similarity with either the heavy or light chain variable domain of the parent antibody. In another embodiment, the antibody variant will have about 80% sequence identity or similarity with either the heavy or light chain variable domain of the parent antibody. In another embodiment, the antibody variant will have about 83% sequence identity or similarity with either the heavy or light chain variable domain of the parent antibody. In another embodiment, the antibody variant will have about 85% sequence identity or similarity with either the heavy or light chain variable domain of the parent antibody. In another embodiment, the antibody variant will have about 87% sequence identity or similarity with either the heavy or light chain variable domain of the parent antibody. In another embodiment, the antibody variant will have about 90% sequence identity or similarity with either the heavy or light chain variable domain of the parent antibody. In another embodiment, the antibody variant will have about 92% sequence identity or similarity with either the heavy or light chain variable domain of the parent antibody. In another embodiment, the antibody variant will have about 95% sequence identity or similarity with either the heavy or light chain variable domain of the parent antibody. In another embodiment, the antibody variant will have about 97% sequence identity or similarity with either the heavy or light chain variable domain of the parent antibody. Identity or similarity with respect to this sequence is defined herein as the percentage of amino acid residues in the candidate sequence that are identical (i.e same residue) with the parent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity. The antibody variant is generally one that has a longer hypervariable region (by one or more amino acid residues; e.g. by about one to about 30 amino acid residues and preferably by about two to about ten amino acid residues) than the corresponding hypervariable region of a parent antibody.

An “amino acid alteration” refers to a change in the amino acid sequence of a predetermined amino acid sequence. Exemplary alterations include insertions, substitutions and deletions.

An “amino acid insertion” refers to the introduction of one or more amino acid residues into a predetermined amino acid sequence The amino acid insertion may comprise a “peptide insertion” in which case a peptide comprising two or more amino acid residues joined by peptide bond(s) is introduced into the predetermined amino acid sequence. Where the amino acid insertion involves insertion of a peptide, the inserted peptide may be generated by random mutagenesis such that it has an amino acid sequence which does not exist in nature.

The inserted residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (lie): leucine (Leu); lysine (Lys); methionine (Met); phenylalanine (Phe); proline (Pro); serine (Ser); threonine (Thr); tryptophan (Trp); tyrosine (Tyr); and valine (Val).

Insertion of one or more non-naturally occurring amino acid residues is also encompassed by the definition of an amino acid insertion herein. A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, omithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) and Eliman et al., supra, can be used. Briefly, these procedures involve chemically activating a suppressor tRNA with a non-naturally occurring amino acid residue followed by in vitro transcription and translation of the RNA.

An amino acid insertion “in a hypervariable region” refers to the introduction of one or more amino acid residues within a hypervariable region amino acid sequence.

An amino acid insertion “adjacent a hypervariable region” refers to the introduction of one or more amino acid residues at the N-terminal and/or C-terminal end of a hypervariable region, such that at least one of the inserted amino acid residues forms a peptide bond with the N-terminal or C-terminal amino acid residue of the hypervariable region in question.

An “amino acid substitution” refers to the replacement of an existing amino acid residue in a predetermined amino acid sequence with another different amino acid residue. Each type of antibody variant described herein represents a separate embodiment of the present invention.

It is to be understood that any peptide of the present invention may, in one embodiment, be isolated, generated synthetically, obtained via translation of sequences subjected to any mutagenesis technique, as well as obtained via any protein evolution techniques, known to those skilled in the art.

In another embodiment, recombinant protein production is a means whereby peptides of the invention are produced. The recombinant proteins may then, in some embodiments, be introduced into an organism. Any method of generating proteins or peptides known in the art represents a separate embodiment of the present invention.

Antibody “binding affinity” may be determined by equilibrium methods (e.g. enzyme-linked immunoabsorbent assay (ELISA) or radioimmunoassay (RIA)), or kinetics. Methods for assessing antibody binding affinity are well known in the art, and are described, for example, in Ravindranath M et al, J Immunol Methods 169: 257-72, 1994; Schots A et al, J Immunol Methods 109: 225, 1988; and Steward M et al, Immunology 72: 99-103, 1991; and Garcia-Ojeda P et al, Infect Immun 72: 3451-60, 2004. Each technique represents a separate embodiment of the present invention.

In another embodiment, the present invention provides an isolated nucleic acid encoding any anti-haptoglobin (Hp) antibody of the present invention. In another embodiment, the present invention provides an isolated nucleic acid encoding any antigen-binding fragment of the present invention.

In one embodiment of the present invention, “nucleic acid” refers to a string of at least two base-sugar-phosphate combinations. The term includes, in one embodiment, deoxyribonucleic acid (DNA) and ribonucleic acid (RNA). “Nucleotide” refers, in one embodiment, to a monomeric unit of a nucleic acid polymer. RNA may be in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) or ribozymes. The use of siRNA and miRNA has been described (Caudy A A et al, Genes & Devel 16:2491-96 (2002), Paddison P J et al., Methods Mol Biol. 265:85-100 (2004), Paddison P J et al., Proc Natl Acad Sci U S A. 99:1443-8 (2002) and references cited therein). DNA may be in the form of plasmid DNA, viral DNA, linear DNA, or chromosomal DNA or derivatives of these groups. In addition, these forms of DNA and RNA may be single, double, triple, or quadruple stranded. The term also includes, in one embodiment, artificial nucleic acids that may contain other types of backbones but the same bases. Examples of artificial nucleic acids are PNAs (peptide nucleic acids), phosphorothioates, and other variants of the phosphate backbone of native nucleic acids. PNA may contain peptide backbones and nucleotide bases, and may be able to bind both DNA and RNA molecules. The use of phosphothiorate nucleic acids and PNA are known to those skilled in the art, and are described in, for example, Nielsen P E, Curr Opin Struct Biol 9:353-57 (1999), Nielsen P E., Mol Biotechnol. 26:233-48 (2004), Rebuffat A G et al., FASEB J. 16:1426-8 (2002), Inui T et al., J. Biol. Chem. 272:8109-12 (1997), Chasty R et al., Leuk Res. 20:391-5 (1996) and references cited therein; and Raz N K et al Biochem Biophys Res Commun. 297:1075-84. In another embodiment, the term includes any derivative of any type of RNA or DNA known in the art. The production and use of nucleic acids is known to those skilled in art and is described, for example, in Molecular Cloning, Sambrook and Russell, eds. (2001), and Methods in Epmology: Guide to Molecular Cloning Techniques (2001) Berger and Kimmel, eds. Each nucleic acid derivative represents a separate embodiment of the present invention.

The nucleic acids can be produced by any synthetic or recombinant process that is known in the art. Nucleic acids can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

DNA according to the invention can also be chemically synthesized by any method known in the art. For example, the DNA can be synthesized chemically from the four nucleotides in whole or in part by methods known in the art. Such methods include those described in Caruthers M H, Science 230:281-5 (1985). DNA can also be synthesized by preparing overlapping double-stranded oligonucleotides, filling in the gaps, and ligating the ends together (see, generally, Molecular Cloning (ibid) and Glover R P et al., Rapid Commun Mass Spectrom 9:897-901, 1995). DNA expressing functional homologues of the protein can be prepared from wild-type DNA by site-directed mutagenesis (see, for example, Molecular Biology: Current Innovations and Future Trends. A. M. Griffin and H. G. Griffin, Eds. (1995); and Kim D F et al, Cold Spring Harb Symp Quant Biol. 66:119-26 (2001). The DNA obtained can be amplified by methods known in the art. One suitable method is the polymerase chain reaction (PCR) method described in Molecular Cloning (ibid). Each of these methods represents a separate embodiment of the present invention.

Methods for modifying nucleic acids to achieve specific purposes are disclosed in the art, for example, in Molecular Cloning (ibid). Moreover, the nucleic acid sequences of the invention can include one or more portions of nucleotide sequence that are non-coding for the protein of interest. Variations in the DNA sequences, which are caused by point mutations or by induced modifications (including insertion, deletion, and substitution) to enhance the activity, half-life or production of the polypeptides encoded thereby, are also encompassed in the invention. Each of these methods and variations represents a separate embodiment of the present invention.

In another embodiment, the present invention provides a vector comprising any nucleic acid of this invention. In another embodiment, the present invention provides a cell or packaging cell line comprising any antibody, peptide, or nucleic acid of this invention. In one embodiment, “vector” refers to a vehicle that facilitates expression of a nucleic acid molecule inserted therein in a cell. In another embodiment, a vector may facilitate expression in an expression system such as a reticulocyte extract. A vector may, in one embodiment, comprise a nucleic acid comprising non-coding nucleic acid sequences or coding sequences other than the inserted nucleic acid.

A large number of vectors known in the art may be used in this embodiment. A vector may include, in some embodiments, an appropriate selectable marker. In other embodiments, the vector may further include an origin of replication, or may be a shuttle vector, which can propagate both in bacteria, such as, for example, E. coli (wherein the vector comprises an appropriate selectable marker and origin of replication) or be compatible for propagation in vertebrate cells, or integration in the genome of an organism of choice. The vector according to this aspect of the present invention may be, for example, a plasmid, a bacmid, a phagemid, a cosmid, a phage, a modified or unmodified virus, an artificial chromosome, or any other vector known in the art. Many such vectors are commercially available, and their use is well known to those skilled in the art (see, for example, Molecular Cloning. (2001), Sambrook and Russell, eds.). Each vector represents a separate embodiment of the present invention.

In another embodiment, the nucleotide molecule present in the vector may be a plasmid, cosmid, or the like, or a vector or strand of nucleic acid. In another embodiment, the nucleotide molecule may be genetic material of a living organism, virus, phage, or material derived from a living organism, virus, or phage. The nucleotide molecule may be, in one embodiment, linear, circular, or concatemerized, and may be of any length. Each type of nucleotide molecule represents a separate embodiment of the present invention.

According to another embodiment, nucleic acid vectors comprising the isolated nucleic acid sequence include a promoter for regulating expression of the isolated nucleic acid. Such promoters are known to be cis-acting sequence elements required for transcription, as they serve to bind DNA-dependent RNA polymerase, which transcribes sequences present downstream thereof. Each vector disclosed herein represents a separate embodiment of the present invention.

In one embodiment, the isolated nucleic acid may be subcloned into the vector. “Subcloning”, in all the applications disclosed herein, refers, in one embodiment, to inserting an oligonucleotide into a nucleotide molecule. For example, in one embodiment isolated DNA encoding an RNA transcript can be inserted into an appropriate expression vector that is suitable for the host cell such that the DNA is transcribed to produce the RNA.

The insertion into a vector can, in one embodiment, be accomplished by ligating the DNA fragment into a vector that has complementary cohesive termini. However, if the complementary restriction sites used to fragment the DNA are not present in the cloning vector, the ends of the DNA molecules may, in another embodiment, be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Methods for subcloning are known to those skilled in the art, and are described, for example in Molecular Cloning (2001), Sambrook and Russell, eds. Each of these methods represents a separate embodiment of the present invention.

“Packaging cell line” refers, in one embodiment, to a cell comprising all or a portion of a viral genome and capable of producing viral particles. In one embodiment, the packaging cell line requires that additional viral sequences be supplied exogenously (for example, in a vector, plasmid, or the like) in order to produce viral particles. In another embodiment, the packaging cell line does not require additional viral sequences to produce viral particles. The construction and use of packaging cell lines is well known in the art, and is described, for example, in U.S. Pat. No. 6,589,763 and Kalpana G V et al, Semin Liver Disease 19:27-37 (1999). Each packaging cell line known in the art represents a separate embodiment of the present invention.

In another embodiment, the present invention provides a method of determining a haptoglobin type of a subject, comprising (a) contacting a biological sample of the subject with an anti-haptoglobin antibody; and (b) quantitatively determining a binding or interaction between the haptoglobin protein and the antibody under conditions whereby a value obtained from the quantitatively determination is characteristic of a presence of Hp 1-1, Hp 2-1, or Hp 2-2 in the biological sample. For example, Hp 1-1, 2-1, and 2-2 produce characteristic values in a sandwich ELISA assay utilizing the E3 antibody of the present invention (Example 2).

In one embodiment, the anti-haptoglobin (Hp) antibody utilized in the method may bind with greater affinity to Hp 2-2 than to Hp 2-1. In another embodiment, the anti-haptoglobin (Hp) antibody utilized in the method may not bind with greater affinity to Hp 2-2 than to Hp 2-1. In one embodiment, the anti-haptoglobin (Hp) antibody utilized in the method may bind with greater affinity to Hp 2-1 than to Hp 1-1. In another embodiment, the anti-haptoglobin (Hp) antibody utilized in the method may not bind with greater affinity to Hp 2-1 than to Hp 1-1. In one embodiment, the anti-haptoglobin (Hp) antibody utilized may have an amino acid sequence as set forth in SEQ ID No 1. In another embodiment, the anti-haptoglobin (Hp) antibody utilized may be any antibody that binds to haptoglobin.

In one embodiment, the method of the present invention may yield a value characteristic of the presence of Hp 1-1, Hp 2-1, or Hp 2-2 over a range of haptoglobin concentrations between about 0.15 grams per liter and about 2.5 grams per liter. In another embodiment, the method of the present invention distinguishes between Hp 1-1, 2-1, and 2-2 over the physiological range of haptoglobin concentration. In another embodiment, the method of the present invention distinguishes between Hp 1-1, 2-1, and 2-2 only over a narrower range of haptoglobin concentration. In one embodiment, the ability of the method of the present invention to distinguish between Hp 1-1, 2-1, and 2-2 is unaffected by hemolysis. In another embodiment, the ability of the method of the present invention to distinguish between Hp 1-1, 2-1, and 2-2 is unaffected by hemolysis. Each method represents a separate embodiment of the present invention.

In one embodiment, the method of the present invention may comprise enzyme-linked immunosorbent assay (ELISA). Methods for ELISA are well known in the art, and are described, for example, in U.S. Pat. No. 5,654,407. In one embodiment of this method, the concentration of antigen is measured using two kinds of monoclonal antibodies which recognize different epitopes of the antigen. In the first stage of this embodiment, an antigen-containing sample is poured on a measurement plate on which antibodies (capture antibodies) have been adsorbed; the antigens in sample are bound to the primary antibodies. In the second stage, the substances in the sample other than the antigen are washed off with a washing agent. Then, in the third stage, a solution of the secondary antibodies, labeled with reporter molecules, such as an enzyme or radioisotope, are poured on the plate; the labeled antibodies bind to the antigens having been bound to the primary antibodies. In one embodiment, the secondary antibodies may have the same specificity as the capture antibodies. In another embodiment, the secondary antibodies may have a different specificity from the capture antibodies. Each type of method represents a separate embodiment of the present invention.

Excessive labeled antibodies are, in one embodiment, fully rinsed away with washing agent, then the amount of the reporter molecules left in the measurement plate is measured by means of an enzyme activity reader or a liquid scintillation counter; and the observed values are used for the estimation of the quantity of antigens in the sample.

In another embodiment, the method of the present invention may comprise a reporter molecule without the use of a capture antibody. Each method represents a separate embodiment of the present invention.

In another embodiment, any method of the present invention may be utilized to test a subject for susceptibility to diabetic complications. In one embodiment, diabetic complications refers to vascular complications. In another embodiment, diabetic complications refers to restenosis after PTCA or coronary artery stent implantation. In another embodiment, diabetic complications refers to diabetic nephropathy. In another embodiment, diabetic complications refers to risk of cardiovascular disease. In another embodiment, diabetic complications refers to mortality in a defined period following acute myocardial infarction. In another embodiment, diabetic complications refers to diabetic cardiovascular disease. In another embodiment, diabetic complications refers to diabetic retinopathy. In another embodiment, diabetic complications refers to any other type of complication of diabetes in which haptoglobin type may play a role. Each diabetic complication represents a separate embodiment of the present invention.

In another embodiment, the present invention provides a method of testing an antibody or recombinant protein for a utility in distinguishing between Hp 1-1, Hp 2-1, and Hp 2-2, comprising (a) contacting a first quantity of the antibody or recombinant protein with an Hp 1-1 molecule; (b) contacting a second quantity of the antibody or recombinant protein with an Hp 2-2 molecule; (c) contacting a third quantity of the antibody with an Hp 2-2 molecule; and (d) quantitatively determining a binding or interaction between the antibody or recombinant protein and the Hp 1-1, Hp 2-1, and Hp 2-2, whereby a value obtained from the quantitatively determination that is characteristic of the presence of each of Hp 1-1, Hp 2-1, or Hp 2-2 indicates that the antibody distinguishes between Hp 1-1, Hp 2-1, and Hp 2-2. In one embodiment of this method, the antibody or recombinant protein may be tested for utility in distinguishing between Hp 1-1, Hp 2-1, and Hp 2-2 when used as the capture antibody in a sandwich ELISA. Any method described herein may be used to test an antibody or recombinant protein for a utility in distinguishing between Hp 1-1, Hp 2-1, and Hp 2-2, and each method represents a separate embodiment of the present invention.

In one embodiment, the antibody may be further tested for an ability to distinguish between Hp 1-1, Hp 2-1, and Hp 2-2 over a range of different haptoglobin concentrations. In another embodiment, the antibody may be tested for an ability to distinguish between Hp 1-1, Hp 2-1, and Hp 2-2 at only a single haptoglobin concentration. Each of these methods represents a separate embodiment of the present invention.

In another embodiment, the present invention provides a method of testing an antibody or recombinant protein for a utility in distinguishing between Hp 1-1, Hp 2-1, and Hp 2-2, comprising (a) immobilizing an anti-haptoglobin antibody on a substrate to form an antibody-substrate complex; (b) contacting a first quantity of the antibody-substrate complex with an Hp 1-1 molecule; (c) contacting a second quantity of the antibody-substrate complex with an Hp 2-1 molecule; (d) contacting a third quantity of the antibody-substrate complex with an Hp 2-2 molecule; (e) contacting the products of steps (b), (c) and (d) with the test antibody or recombinant protein; and (i) quantitatively determining a binding or interaction between the test antibody or recombinant protein and the Hp 1-1, Hp 2-1, and Hp 2-2; whereby a value obtained from the quantitatively determination that is characteristic of the presence of each of Hp 1-1, Hp 2-1, or Hp 2-2 indicates that the test antibody distinguishes between Hp 1-1, Hp 2-1, and Hp 2-2.

In one embodiment, the antibody may be further tested for an ability to distinguish between Hp 1-1, Hp 2-1, and Hp 2-2 over a range of different haptoglobin concentrations. In another embodiment, the antibody may be tested for an ability to distinguish between Hp 1-1, Hp 2-1, and Hp 2-2 at only a single haptoglobin concentration. Each of these methods represents a separate embodiment of the present invention.

In another embodiment, the present invention provides a method of screening a plurality of test antibodies for an ability to differentially interact with different haptoglobin types, comprising (a) generating a plurality of vehicles, each comprising an antibody from the plurality of test antibodies and a nucleic acid molecule encoding the antibody; (b) contacting the plurality of vehicles with non-immobilized Hp 1-1 or Hp 2-1 and Hp 2-2 that is immobilized on a substrate; (c) subcloning a nucleic acid molecule from one of the plurality of vehicles into a vehicle that expresses an antibody encoded by the nucleic acid molecule; (d) repeating steps (b) and (c) one or more times; and (e) identifying an antibody or nucleic acid molecule present in a vehicle retained on the substrate. In one embodiment, the antibodies utilized in the method may be single chain Fv (scFv) antibodies. The present invention shows the screening of an antibody to identify the E3 scFv antibody by this method (Example 1).

In one embodiment, the plurality of test antibodies screened is generated in an animal lacking an Hp 2-2 allele. Use of mice, an animal lacking an Hp 2-2 allele (Example 1) may, in one embodiment, favor the generation of antibodies that preferentially bind Hp 2-2 over Hp 2-1.

In one embodiment, the vehicle may be a phage or virus. In another embodiment, the vehicle may be any vehicle capable of carrying an antibody and a nucleic acid molecule encoding the antibody. Each method represents a separate embodiment of the present invention.

In one embodiment, the subcloning of step (c) of the method results in an amplification of a nucleic acid molecule encoding an antibody with an ability to differentially interact with different haptoglobin types.

In another embodiment, the present invention provides a method of distinguishing between two allelic variants of a polymorphic protein in a biological sample, wherein the two allelic variants differ in a number of copies of an epitope, comprising (a) contacting a biological sample with an antibody or recombinant protein, wherein the antibody or recombinant protein binds the polymorphic protein; and (b) quantitatively assessing a binding or interaction between the polymorphic protein and the antibody or recombinant protein; under conditions whereby the presence of each of the two allelic variants results in a value obtained from the quantitatively assessing that is characteristic of the allelic variant. The present invention provides the first demonstration that allelic variants of a polymorphic protein that differ solely in a number of copies of an epitope may nevertheless be differentiated on the basis of antibody reactivity (Example 2).

In one embodiment, the antibody or recombinant protein used in the method may differentially bind the allelic variants. In another embodiment, the recombinant protein may have the same intrinsic affinity for the allelic variants. Any method of the present described herein may be used for distinguishing allelic variants of any polymorphic protein, in a manner analogous to the applications for haptoglobin described herein. Each method represents a separate embodiment of the present invention.

Without wishing to be bound by theory, the difference observed between the reactivity of Hp 1-1 and Hp 2-2 in the sandwich ELISA may be attributable to the fact that Hp 1-1 dimers have only 2 antigenic sites recognized by E3, while Hp 2-2 polymers have 3 or more antigenic sites. Binding of both sites of a dimer to an immobilized E3 antibody may thus prevent binding of second (detection) E3 antibody. According to this embodiment, such a blocking event by the first capture antibody is less likely to occur as the number of polymeric units in the Hp protein increases hence giving rise to a greater signal when using Hp 2-1 and an even greater signal with Hp 2-2. Thus, based on the present invention, the sandwich ELISA method will be useful in distinguishing allelic variants of any polymorphic protein, in a manner analogous to the applications for haptoglobin described herein.

In another embodiment, the present invention provides a kit that comprises any method of determining a haptoglobin type of a subject, method of testing a subject for susceptibility to diabetic complications, method of testing an antibody or recombinant protein for a utility in distinguishing between Hp 1-1, Hp 2-1, and Hp 2-2, or method of distinguishing between two allelic variants of a polymorphic protein in a biological sample described in the present invention. Kits are packages that facilitate a diagnostic or other procedure by providing materials or reagents needed thereof in a convenient format. Many kits have been successfully commercialized.

In one embodiment, the kit may further comprise an apparatus for performing enzyme-linked immunosorbent assay (ELISA). In another embodiment, the kit may not comprise an apparatus for performing enzyme-linked immunosorbent assay (ELISA). Each type of kit represents a separate embodiment of the present invention.

In another embodiment, the present invention provides a composition comprising an isolated nucleic acid, polypeptide, vector, cell, or packaging cell line of this invention. In one embodiment, the composition may comprise a liposome or other vehicle for introducing the isolated nucleic acid into a cell or for introducing the nucleic acid into a patient.

In another embodiment, routes of administration of the nucleic acids, vectors, peptides, compounds and compositions of the invention include, but are not limited to oral or local administration, such as by aerosol, intramuscularly or transdermally, and parenteral application. Compositions can be administered in a variety of unit dosage forms depending upon the method of administration. Suitable unit dosage forms, include, but are not limited to powders, tablets, pills, capsules, lozenges, suppositories, etc. Transdermal administration may be accomplished by application of a cream, rinse, gel, etc. capable of allowing the active compounds to penetrate the skin. Parenteral routes of administration may include, but are not limited to, electrical or direct injection such as direct injection into a central venous line, intravenous, intramuscular, intraperitoneal, intradermal, or subcutaneous injection.

In another embodiment compositions of the present invention may include, but are not limited to: suspensions, oils, creams, and ointments applied directly to the skin or incorporated into a protective carrier such as a transdermal device (“transdermal patch”). Examples of suitable creams, ointments, etc. can be found, for instance, in Physician's Desk Reference (2003) Gruenwald, ed. Examples of suitable transdermal devices are described, for instance, in U.S. Pat. No. 4,818,540.

In another embodiment, compositions of the present invention suitable for parenteral administration include, but are not limited to, sterile isotonic solutions. Such solutions include, but are not limited to, saline and phosphate buffered saline for injection into a central venous line, intravenous, intramuscular, intraperitoneal, intradermal, or subcutaneous injection.

### EXAMPLES

### Example 1

### Isolation and Identification of an scFv Antibody that Preferentially Binds Hp 2-2 Over Hp 1-1

Immunization

C57B1/6mice were immunized with an emulsion containing purified protein-derived peptide of tuberculin (PPD) covalently coupled with human Hp 2-2 protein, as described (Andersen, P et al, Proc. Natl. Acad. Sci. USA 93: 1820, 1996). Mice were initially immunized subdermally and subsequently subcutaneously at 2-week intervals for a period of 3-5 months with 2030 microgram (μg) per mouse of the antigenic mixture in Incomplete Freund's Adjuvant. Mice were sacrificed and spleens collected 2 weeks after the last immunization.

Library Construction

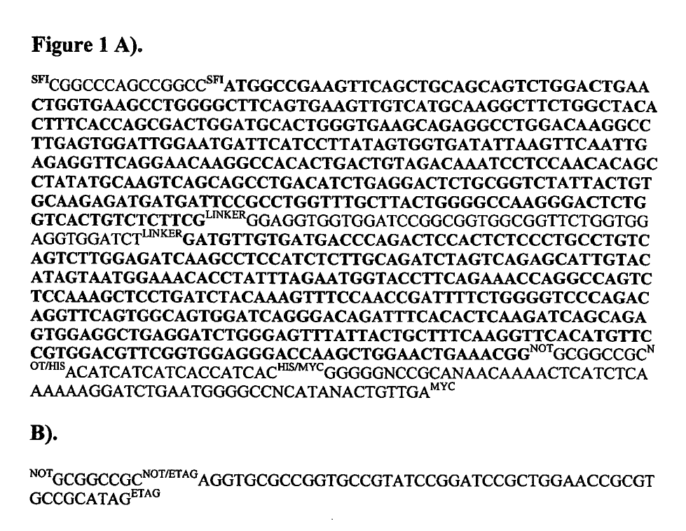

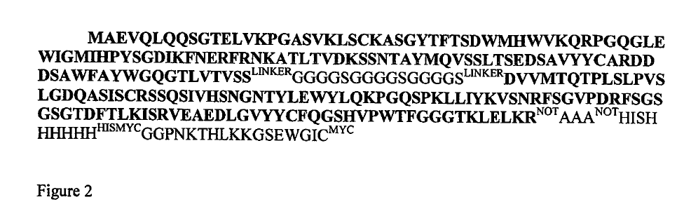

The scFv repertoire was prepared by amplifying mRNA from the spleen tissue by reverse transcripase polymerase chain reaction (RT-PCR) (Benhar I et al, Curr. Protocols Immunol 48: 59, 2002). Sfi I-Not I fragments of the RT-PCR products (FIG. 1A) were subcloned into the pCANTAB6 phagemid vector (Berdichevsky, Y et al, J. Immunol. Methods 228: 15, 1999) to generate a library of phages, each displaying a clone of C-terminally myc-tagged scFv antibody. The library contained 1.5×10⁶ independent clones. The amino acid sequence of E3 is depicted in FIG. 2.

Antibody Selection

Clones binding Hp 2-2 were selected by incubating 10¹¹ colony-forming units (cfu) from the library in immunotubes (Nunc) coated with Hp 2-2 protein. After extensive washing, bound phages were eluted with triethylamine. E. Coli TG1 cells were infected with the eluted phages, then superinfected with M13KO7 helper phage to amplify the genome of the eluted phages (Berdichevsky Y et al, J. Immunol. Methods 228: 151, 1999).

This panning process was repeated 6 times, with Hp 1-1 present in the immunotube incubation buffer during rounds 4, 5 and 6 in order to select for phage clones containing antibody that bound Hp 2-2 with significantly greater affinity than Hp 1-1.

Subsequently, individual phage clones were screened for ability to differentially bind immobilized Hp 2-2 and Hp 1-1 in an ELISA assay. Phage clone E3 bound immobilized Hp 2-2 significantly better than Hp 1-1. The myc-tagged single chain E3 antibody was then purified, and its affinity for Hp 1-1 or Hp 2-2 immobilized in a plastic microwell was tested in a ELISA assay, using horseradish peroxidase (HRP)-conjugated anti-myc as the detection antibody. A 4-fold greater signal was obtained with Hp 2-2 compared to Hp 1-1.

### Example 2

### The E3 antibody can distinguish between Hp 1-1, 2-1, and 2-2 in an ELISA sandwich assay

### Materials and Experimental Methods

Microtiter plates (Maxisorb, Nunc) were coated with 100 microliter/well of E3-Etag antibody (10 μg/ml in coating buffer) overnight at 4° C. Wells were washed with Tris-buffered saline (TBS) containing 0.05% Tween and then incubated with 150 μl/well blocking buffer (TBS with 1% BSA and 0.1% Tween) for 1-2 hours at 37° C. Serum samples diluted 1:100 in blocking buffer were added to the wells (100 μl) and incubated for 1 hour at room temperature (RT). After washing, 100 μl/well E3-myc antibody was s added at a concentration of 0.8 microgram (μg)/ml and the plates incubated for 1 hour at RT. After washing, HRP conjugated anti-myc antibody (diluted 1:1000) was added and the plates incubated for 1 hour at RT. Plates were developed with TMB substrate (DAKO) and quenched with 100 μl/well 1 normal sulfuric acid. Quantitation was performed by measuring absorbance at 450 nm.

### Results

The assay was modified by using E3 as both the capture antibody and the detection antibody in a sandwich ELISA. An E3 antibody lacking a myc tag was generated by subcloning a Sfi I-Not I fragment of E3 into the pCANTAB5E vector (FIG. 1B), and was used as the detection antibody, while using the myc-tagged E3 antibody as the capture antibody as before. The E protein tag was not necessary, but rather was introduced because it was present in the pCANTAB5E vector.

Sera from individuals with Hp 1-1, 2-1, or 2-2 (three individuals per group) were analyzed. Absorbance readings were 0.196±0.007, 0.560±0.033 and 0.916±0.009 respectively. These results demonstrate that the E3 antibody can distinguish between the 1-1,2-1 and 2-2 forms of Hp in a sandwich assay.

### Example 3

### A Sandwich Assay Utilizing the E3 Antibody Distinguishes Between Hp 1-1, 2-1, and 2-2 Over the Physiological Range of Haptoglobin Concentration and is Unaffected by Hemolysis

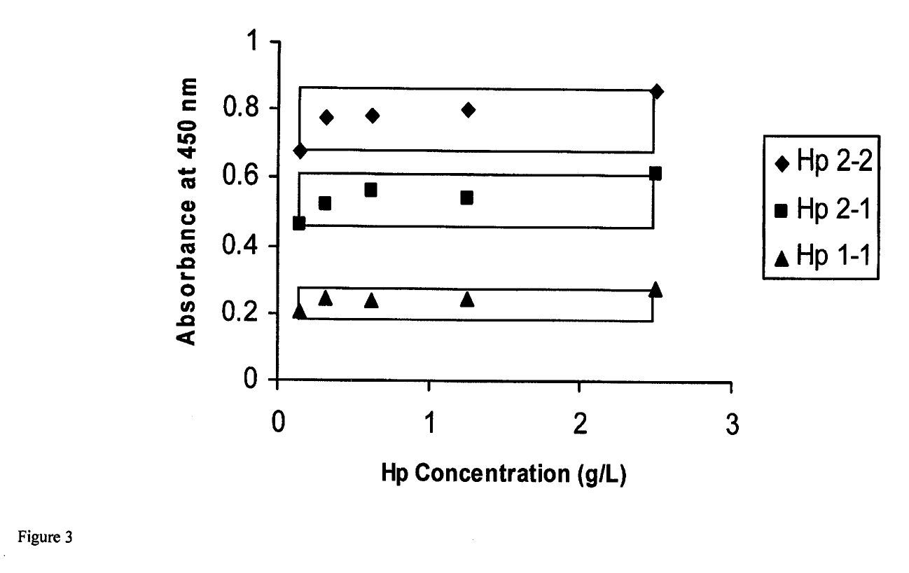

The normal range of Hp in serum is 0.3 to 2.0 g/L in Caucasians and 0.12-2.15 g/L in Zimbabwean Blacks. In order to test the sandwich assay utilizing the E3 antibody over this range of concentrations, serum was depleted of haptoglobin by passage over a hemoglobin-agarose column, and then haptoglobin 1-1, 2-1 or 2-2 was added back at concentrations ranging from 0.15 to 2.5 g/L. ELISA analysis showed that the absorbance at 450 nm for the three Hp types was distinguishable over this range of Hp concentrations, demonstrating that the sandwich assay utilizing the E3 antibody can distinguish between haptoglobin 1-1, 2-1 and 2-2 over the normal physiological range of haptoglobin concentration (FIG. 3).

Hemoglobin was added to serum samples at a concentration of 14 mg/ml, which is a 10-fold excess of serum haptoglobin, resulting in binding of all haptoglobin by hemoglobin and therefore mimicking the effect of complete hemolysis. No effect on absorbance at 450 mn by the excess hemoglobin was observed, demonstrating that the assay is not sensitive to hemolysis.

### Example 4

### The Sandwich Assay Utilizing the E3 Antibody Corresponds with the Gel Electrophoresis Method for Assigning an Hp Type

### Materials and Experimental Methods

Gel Electrophoresis of Hp Type Determination

Polyacrylamide gel electrophoresis was performed on Hb-supplemented serum, followed by visualization of Hp-Hb bands by staining the gel with metal-enhanced peroxidase reagents (Pierce Corp.) as described in Hochberg I et al, Atherosclerosis 161: 441-446,2002.

### Results

In order to test the diagnostic accuracy of the ELISA method for haptoglobin phenotyping, serum samples from 508 individuals (70 Hp 1-1, 224 Hp 2-1, 2 Hp 2-1M, and 214 Hp 2-2), who had previously been typed by protein gel electrophoresis, were analyzed by the ELISA method. Each assay included three samples of each of the major haptoglobin phenotypes as standards. An average absorbance was calculated for each phenotype. Cutoff values were assigned at the midway point between the different phenotypes. Samples that fell on the borderline between two phenotypes were repeated in order to confirm haptoglobin type.

There was a 96% correspondence between the ELISA method and the gel etectrophoresis method. The error rate was independent of haptoglobin phenotype. Incorrect assignment was noted in those serum samples of the 2-1M phenotype (0.4%) and in samples whose haptoglobin concentration fell below 0.15 g/L, as evidenced by a faint or partially degraded banding pattern following gel electrophoresis. These findings demonstrate that this method can distinguish between different haptoglobin types.

### Example 5

### Obtaining Antiserum Specific for the Haptoglobin (Hp) 2 Allelic Protein product that does Not Cross React with the Hp1 Allelic Protein Product

Method

Immunize rabbits or mice with a peptide from the junction between exons 4 and 5 of Hp 2 protein—this junction is the only epitope unique to Hp2 based on the primary amino acid sequence of the Hp 1 and Hp 2 proteins.

The exon 4/5 junction peptide (20 amino acids) was cloned as a PCR fragment first into the vector pTeasy (Promega Biotec) and then as a Bam/EcoR1 fragment into the vector pGEX-2TK (Pharmacia/Danylel Biotech). The resulting plasmid encodes a fusion protein between the enzyme glutathione-S-transferase (GST) and the 4/5 junction peptide.

Sequence of PCR primers used to clone the 4/5 junction fragment:

| | |
|---|---|
| Sense: | |
| 5′ - CGC GGA TCC GTT GGA GAT AAA CTT | (SEQ ID NO.4) |
| CCT GAA TGT -3′ | |
| Antisense: | |
| 5′ - GCG GAA TTC TTA AAT CTC GGG GGG | (SEQ ID NO.5) |
| CTT CGG GCA GCC -3′ | |

Nucleotide sequence of cloned PCR fragment in pTeasy vector:

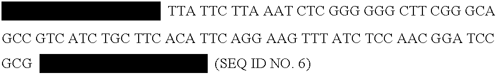

Note:
shaded regions indicate pTeasy vector sequences.

A Bam/EcoR1 fragment from the pTeasy recombinant was then subcloned into pGEX-2Tk and transformed into E. coli strain BL21. A fusion protein of approximately 35 Kd was purified from the periplasmic fraction of IPTG induced bacteria representing the junction peptide fused to GST. This fusion protein was then used to prepare antiserum in either mice or in rabbits (polyclonal). This antiserum as demonstrated below was then tested for its ability to differentiate between the Hp 1-1, Hp 2-1 and Hp 2-2 proteins in an ELISA format.

Results

ELISA Results Using Antifusion Peptide Antiserum

ELISA plates were coated with 10 ug/ml Hp (Hp 1-1, Hp 2-1 or Hp 2-2 as indicated). Anti peptide antiserum was used at 1:1000 dilution. Secondary antibodies were goat anti-mouse or goat anti rabbit HRP conjugated Abs as appropriate.

Results Using Mouse Antiserum

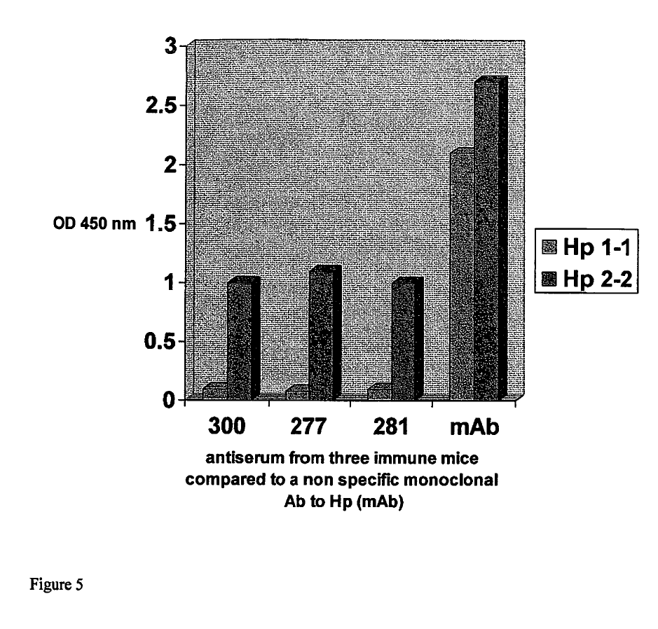

(OD450 refers to HRP signal from secondary antibody; numbers (300, 277, 281 refer to antiserum taken from three different mice), results are shown in FIG. 5, indicating that the mouse fusion protein antiserum can easily distinguish Hp 1-1 from Hp 2-2.

Results Using Rabbit Anti-Fusion Peptide Antiserum

Unpurified or purified-purified antiserum was made by first passing the crude antiserum over a GST column allowing all antiserum with specificity for GST to be depleted from the antiserum. The flow thru was then reapplied to a GST-peptide column and the antibody binding to this GST-peptide was then eluted and used for these studies.

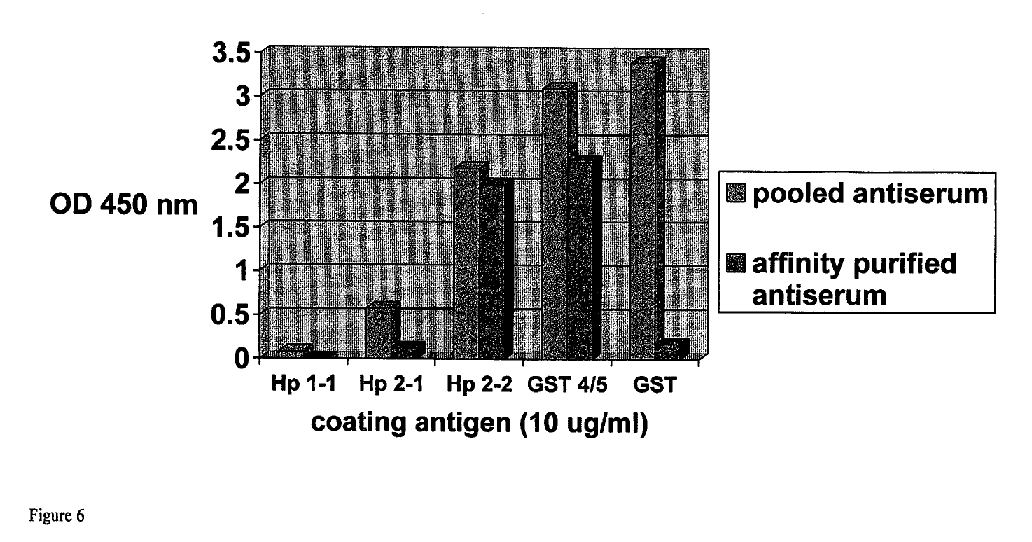

As shown in FIG. 6, both the crude and affinity purified 4/5 junction peptide antiserum from rabbits can differentiate Hp I-1, Hp 2-1 and Hp 2-2 in an ELISA format. GST 4/5 is the fusion protein. GST is GST without the junction peptide. Note GST alone was recognized only by the crude pooled antiserum and not by the affinity purified antiserum.

1-89. (canceled)

90. An anti-haptoglobin (Hp) antibody represented by the amino acid sequence set forth in SEQ ID NO-1, or an antigen binding fragment thereof.

91. The anti-haptoglobin (Hp) antibody of claim 90, having a binding affinity in decreasing order to Hp 2-2; Hp 2-1; and Hp 1-1 alleles of haptoglobin (Hp), wherein said anti-haptoglobin (Hp) antibody is

92. The anti-haptoglobin (Hp) antibody of claim 90, wherein said antibody is a monoclonal antibody.

93. The anti-haptoglobin (Hp) monoclonal antibody of claim 92, wherein said monoclonal antibody is a single chain Fv (scFv) antibody.

94. A composition comprising: the anti-haptoglobin (Hp) antibody of claim 90 or an antigen binding fragment thereof.

95. A composition comprising a cell, a packaging cell line, an antibody, a recombinant protein, or a recombinant viral particle comprising the anti-haptoglobin antibody of claim 90 or an antigen binding fragment thereof.

96. The antibody of claim 95, wherein said antibody is a monoclonal antibody (mAb), a humanized antibody, a chimeric antibody, or a combination thereof.

97. The antibody of claim 96, wherein said monoclonal antibody is a single chain Fv (scFv) antibody.

98. An isolated nucleic acid encoding the anti-haptoglobin (Hp) antibody of claim 90 or an antigen binding fragment thereof.

99. A method of determining a haptoglobin allele type (Hp) of a subject, comprising the steps of contacting a biological sample of said subject with an anti-haptoglobin antibody, forming a bound anti-haptoglobin antibody-antigen complex; quantitatively determining a binding affinity between said biological sample and said anti-haptoglobin antibody; and comparing said quantitatively determined binding affinity with a value obtained from a quantitatively determined binding affinity of said anti-haptoglobin antibody to an isolated Hp 1-1, Hp 2-1, or Hp 2-2, at concentrations between about 0.15 g/L and about 2.5 g/L, wherein the binding affinity determined is indicative of the Hp allele type, thereby determining the allele type of Hp in said subject.

100. The method of claim 99, wherein said anti-haptoglobin antibody is the antibody of claim 90, or an antigen binding fragment thereof

101. The method of claim 99, further comprising immobilizing the bound anti-haptoglobin antibody-antigen complex on a substrate.

102. The method of claim 101, further comprising the steps of contacting said immobilized complex with an additional quantity of said anti-haptoglobin antibody; and determining a binding affinity between said antigen and said additional quantity of said anti-haptoglobin antibody.

103. The method of claim 102, wherein said additional anti-haptoglobin antibody is the anti-haptoglobin antibody of claim 90, or an antigen binding fragment thereof.

104. A method of testing a subject for susceptibility to a diabetic complication, comprising the step of determining the subject's haptoglobin allele type according to the method of claim 99, wherein the presence of Hp 2-2 indicates higher susceptibility to a diabetic complication.

105. The method of claim 104, wherein said diabetic complication is a vascular disease, a nepluopathy, a retinopathy, a cardiovascular disease or a combination thereof.

106. A method of testing an antibody or a recombinant protein for a utility in distinguishing between haptoglobin allele types Hp 1-1, Hp 2-1, or Hp 2-2, comprising the steps of: contacting the antibody or recombinant protein with a known concentration of isolated Hp 1-1 molecule, Hp 2-1 molecule and Hp 2-2 molecule, forming a complex; and quantitatively determining a binding affinity between said antibody or recombinant protein and said Hp 1-1, Hp 2-1, and Hp 2-2; whereby a significantly different binding affinity to each of Hp 1-1, Hp 2-1, and Hp 2-2 indicates that said antibody or recombinant protein is capable of distinguishing between Hp 1-1, Hp 2-1, and Hp 2-2.

107. The method of claim 106, further comprising immobilizing the complex of said Hp 1-1, Hp 2-1, or Hp 2-2 and said antibody or recombinant protein.

108. The method of claim 107, further comprising the steps of contacting said complex with an additional quantity of said antibody or recombinant protein subsequent to said immobilizing; and determining a binding affinity between said said Hp 1-1, Hp 2-1, or Hp 2-2 and said additional quantity of said antibody or recombinant protein.

109. The method of claim 108, wherein said additional antibody is the antibody of claim 90, or an antigen binding fragment thereof.

110. A method of screening a plurality of test antibodies for an ability to differentially interact with different haptoglobin allele types, comprising the steps of: generating a plurality of vehicles, each vehicle comprising an antibody from said plurality of test antibodies and a nucleic acid molecule encoding said test antibody; contacting said vehicles with Hp l-1, Hp 2-1 or Hp 2-2 allele that is immobilized on a substrate, forming an immobilized vehicle-Hp allele complex; subcloning a nucleic acid molecule from one of said immobilized vehicles into a vehicle that expresses an antibody encoded by said nucleic acid molecule; repeating said steps of contacting and subcloning one or more times; and identifying an antibody or nucleic acid molecule present in a vehicle retained on said substrate, thereby screening a plurality of test antibodies for an ability to differentially interact with different haptoglobin types.

111. The method of claim 110, whereby said plurality of test antibodies is generated in an animal lacking the Hp 2-2 allele.

112. The method of claim 110, wherein said vehicle is a phage or virus.

113. The method of claim 110, whereby said antibodies are monoclonal antibodies, single chain Fv (scFv) antibodies, or a combination thereof.

114. The method of claim 110, whereby said step of subcloning results in an amplification of a nucleic acid molecule encoding an antibody with an ability to differentially bind to different haptoglobin (Hp) allele types.

115. A complementarity-determining region of an anti-haptoglobin (Hp) antibody that binds with greater affinity to a first haptoglobin isoform than to a second haptoglobin isoform.

116. A composition comprising the complementarity-determining region of claim 115.

117. An antibody or recombinant protein comprising the complementarity-determining region of claim 115.

118. A composition comprising the antibody or recombinant protein of claim 117.

119. The antibody of claim 117, wherein said antibody is a humanized antibody, a chimeric antibody, or a combination thereof.

120. The antibody of claim 117, wherein said antibody is a monoclonal antibody, a single chain Fv (scFv) antibody or a combination thereof.

121. A cell, packaging cell line, or recombinant viral particle comprising the complementarity-determining region of claim 115.

122. A composition comprising the cell or packaging cell line of claim 121.
